(19) United States
(12) Patent Application Publication
(10) Pub. No.: US 2009/0287063 A1
(43) Pub. Date: Nov. 19, 2009

(54) Hygrometric Determination of Hot Flashes

(76) Inventors: Robert R. Freedman, Ann Arbor, MI (US); Samuel A. Wasson, Livonia, MI (US)

Correspondence Address: Benita J. Rohm;Rohm & Monsanto, 12 Rathbone Place, Grosse Pointe, MI, 48230, US

(21) Appl. No.: 12/085,968

(22) PCT Filed: Nov. 29, 2006
(86) PCT No.: PCT/US06/45707

§ 371 Date: Jun. 2, 2008

Related U.S. Application Data
(60) Provisional application No. 60/741,376, filed on Dec. 1, 2005.

(51) Int. Cl. A61B 5/00

(52) U.S. Cl. 600/300

(57) ABSTRACT

A method of measuring hot flashes based on the sensing of skin moisture incorporates a chip for sensing humidity. The method incorporates a chip for sensing humidity, and a RISC micro-controller. A single tiny low-power device of about the size of a quarter is embedded into a reusable plastic housing. Inexpensive and reliable objective measurement of hot flashes is achieved, along with advantages in size, weight, and extended durations of recording and data analysis periods. Hot flashes are measured as a biomarker of the efficacy of clinical intervention in relieving symptoms of menopause.

### RELATIONSHIP TO OTHER APPLICATION

This application claims the benefit of the filing date of Provisional Patent Application Ser. No. 60/741,376 filed Dec. 1, 2005.

### BACKGROUND OF THE INVENTION

1. Field of the Invention

This invention relates generally to a method of determining the occurrence of hot flashes based on the sensing of skin moisture, and more particularly, to a method that incorporates a transducer that produces electrical signals responsive to humidity.

2. Description of the Related Art

Hot flashes occur in about 80% of women in Western societies. While hot flashes are effectively treated by hormone replacement therapy, the adverse side-effects of this once frequently-prescribed therapy now preclude its use. This has prompted the investigation of a wide variety of alternative therapies. However, there is currently no cost-effective means for objectively measuring the effects of these therapies in the field. There is a substantial and pressing need for the development of an instrument that can meet these requirements.

The current standard methodology for objectively measuring hot flashes is skin conductance level measured at the sternum. Changes in the skin conductance level due to sweating reliably correspond to hot flashes. Despite its sensitivity, however, there are a number of shortcomings of sternal skin conductance level that hinder its use in clinical trials. Primary among these is the need for electrodes and gel that are a substantial burden to the subject and investigator.

Although hormone replacement therapy is the most effective validated treatment for hot flashes, recent findings from the Women's Health Initiative have discouraged its use due to adverse effects. Due to these events, there is substantial interest in the development of new treatments.

A wide variety of therapies are currently under investigation including new pharmaceuticals, botanical preparations, acupuncture, and biofeedback. Most of these investigations will incorporate diary methods as the major outcome measure. There are several problems with diaries. Patient noncompliance and false compliance is a major source of error and bias. Furthermore, hot flashes occurring during sleep are not accurately reported because recall of these events is often poor and most hot flashes do not produce full awakenings. Finally, placebo effects as large as 40-50% occur with self-report. These factors make self-report a poor measure of therapy effectiveness.

The etiology and mechanism of hot flashes remain incompletely understood. Future studies of hormonal and neurologic systems may provide promising leads to improve our understanding of the basic phenomenon and perhaps also shed light on the placebo effect. However, this is likely a complex undertaking. Critical to this effort is the ability to reliably identify when a hot flash has occurred. The leading objective measure in use today, sternal skin conductance monitoring, has some limitations in ambulatory settings. However, a more severe limitation is the inability of sternal skin conductance to provide any information on duration, intensity, and interference with activities. Ultimately, researchers desire a convenient and cost-effective sensor for monitoring hot flashes without cumbersome electrodes that might become compromised if a subject experiences extensive sweating or takes a shower and one that can capture data continuously for relatively long periods of observation. However, researchers also need well-characterized methods for collecting self-reported data. If the primary concern is helping women with hot flashes find relief, then subjective measures collected through diaries or interviews cannot be dismissed. Given the importance of this information, it would make sense to undertake methodologic research to ensure that the best possible systems are used to collect valid and reliable information.

The factors desired to be measured with respect to hot flashes are likely to change over time as more is learned about the underlying phenomenon. This will probably be an evolutionary process, one involving decisions about what biological factors will be most useful for the task at hand, what technologies might be available or easily adaptable, which measures should be bundled together to maximize the precision of data collected with the available technology, and the analysis of the data to generate new hypotheses and perhaps the need for new measurement tools.

Investigators face several challenges when considering the design of studies of hot flashes. Substantial placebo effects and small sample sizes have produced studies with equivocal findings. The placebo effect, while remarkable in its dimensions in some studies of hot flash interventions, is not understood. Distinguishing placebo effects from the natural dissipation of symptoms over time would be extremely helpful. Similarly, the ability to induce a placebo effect to reduce the discomfort and annoyance associated with hot flashes might be helpful. The use of neuroimaging technology offers potential for greater understanding of the placebo effect.

Improvements in the measures of hot flashes require improved knowledge in several areas. These include, for example:
Physical processes underlying hot flashes, which will identify additional factors to measure and the factors that influence the perception and reporting of hot flashes.
Improved sternal skin conductance systems, with additional tools to be developed when other factors of hot flashes are identified.
The performance characteristics of questionnaires and diaries to collect self-reported data on hot flash frequency.
Improved and validated instruments for collecting data on intensity and interference with daily activities.
The mechanism(s) of action of placebo, which may also help distinguish natural attrition of symptoms from placebo effect.
Animal models to elucidate triggers and mechanisms of hot flashes and to screen potential treatments. Investigators interested in studying hot flashes face complex issues.

The incomplete understanding of the basic physiology underlying hot flashes clearly calls for further work in this area. It is understood, however, that some mechanistic studies cannot be conducted with human subjects. Thus, animal models are needed. Animal models could be particularly helpful for understanding the neurobiology of hot flashes and perhaps placebo effects. Scientific advances are being made increasingly at the interfaces of traditional disciplines, and approaches to science are becoming more integrative.

It is likely that a multidisciplinary approach to hot flash research would be helpful given the number of physiologic, clinical, and behavioral factors involved. For example, psychologists and sociologists could contribute to identifying factors that may influence the placebo effect, such as pill color; developing and validating questionnaire items and diary formats; ascertaining the effect of mode of data collection on the quality of the resulting data; and determining the best ways to provide information to subjects. However, if they were part of a multidisciplinary team that included basic scientists, clinicians, and bioengineers, different questions might be asked, and better tools might be developed to collect both subjective and objective data on hot flashes.

The increasing emphasis on collaborative science is also embraced at the NIH level. Since May 2002, the NIH has been engaged in a series of activities collectively known as the “NIH Roadmap,” whose goal, in keeping with the NIH mission of uncovering new knowledge about the prevention, detection, diagnosis, and treatment of disease and disability, is to accelerate both the pace of discovery in these key areas and the translation of therapies from bench to bedside. The timing of this workshop to assess measures of hot flashes appears auspicious for several reasons. First, the issue of refining and validating self-reported measures of symptoms through the use of biomarkers and multidisciplinary research teams is consonant with an NIH Roadmap initiative. Second, the new National Institute for Biomedical Imaging and Bioengineering at the NIH offers impetus for linking biomedical, social, and behavioral scientists with bioengineers to assess and improve existing technology or develop new technologies to collect data on physiological markers specific to hot flashes. Third, people are already purchasing and using CAM modalities or are resuming hormone therapy for relief of hot flashes, and they and their clinicians are eager for and deserve more information on the safety and efficacy of these remedies.

It is, therefore, an object of this invention to provide a method of determining objectively the occurrence of a hot flash.

It is another object of this invention to provide a method of determining objectively the occurrence of a hot flash wherein the recall of the patient is not required in the collection of the data.

It is also an object of this invention to provide a method of determining objectively the effects of therapies in the treatment of hot flashes.

It is a further object of this invention to provide a method of determining objectively the occurrence of a hot flash wherein the need for electrodes and gel is obviated.

It is additionally an object of this invention to provide a method of determining objectively the occurrence of a hot flash wherein data is accurately collected and analyzed without requiring the patient to be awakened.

It is yet a further object of this invention to provide an arrangement that is inexpensive and reusable for sensing the occurrence of a hot flash.

It is also another object of this invention to provide an arrangement for sensing the occurrence of hot flashes and collecting data in relation thereto for an extended period of time.

It is yet an additional object of this invention to provide an arrangement for sensing the occurrence of hot flashes and collecting data that can readily be transferred to a computer for analysis.

### SUMMARY OF THE INVENTION

The foregoing and other objects are achieved by this invention which provides method of determining the presence of a hot flash of a patient, the method comprising the step of monitoring relative humidity. In accordance with a specific illustrative embodiment of the invention, the step of monitoring relative humidity is performed in the vicinity of the sternum of a patient.

A transducer is installed in the vicinity of the sternum of a patient, the transducer having an output port for producing electrical data responsive to relative humidity. In one specific illustrative embodiment of the invention, the transducer is a miniature capacitance transducer.

The electrical data that is recorded in a data recorder, and analyzed to determine a rate of change of the relative humidity. The presence of a hot flash in the patient is determined in response to the rate of change of the relative humidity corresponding to an increase of approximately 3% per minute.

In accordance with an apparatus aspect of the invention, there is provided a transducer that measures relative humidity. In one embodiment, the transducer is a miniature capacitance that is configured to have low weight and small size. In one specific illustrative embodiment of the invention, the capacitance transducer is dimensioned approximately 1.5 inches along a major axis. In other embodiments, the transducer incorporates a chip for sensing humidity, and a RISC micro-controller.

A computer is provided for downloading hot flash event waveforms obtained from the transducer. Additionally, a software system is employed to determine a time rate of change of the hot flash event waveforms obtained from the transducer.

The present invention constitutes a hygrometric (humidity based) alternative to the traditional measurement of hot flashes through sternal skin conductance level. The new transducer measures the relative humidity near the sternum using a capacitive transducer SHT-10, Sensitron. The inventors herein have determined that skin moisture is more directly related to the physiological response than skin conductance level and, therefore, should be at least as sensitive to hot flashes as skin conductance level.

The microcontroller can be programmed to analyze automatically each hot flash event, thereby further reducing the storage and data analysis requirements. The devices can be configured through serial communications, thereby providing choice of measurement parameters.

The inventors herein have developed an objective physiological marker of hot flashes. Skin conductance level is recorded from the sternum using surface electrodes. The electrodes are filled with 0.5 M KCl gel because the salinity of commercial EGG gel is too high and hydrates the sweat glands. The sternum is the preferred recording site because sweating occurs there during most hot flashes and not during emotional stimuli, noise, and movement. A criterion skin conductance level change of 2 μmho (electrical unit of conductance) in 30 seconds was found to correspond with 95% of patient self-reports (event marks) when recorded in the laboratory. This finding was independently replicated in a study showing that the addition of other physiological measures, such as finger temperature and pulse volume, did not improve the predictive value of sternal skin conductance level.

To be useful as an outcome measure, however, skin conductance level should be recorded over extended periods in the patient's natural environment. This has so far proved exceedingly difficult using commercially available systems.

The first system was the Oxford Medilog 4-channel tape cassette recorder and PB2 playback unit (Cephalon A/S, Denmark): The event marker and skin conductance level channel were constructed by the inventors. Twelve-hour recordings were made in 11 symptomatic postmenopausal women. There was a concordance of 86% between the criterion skin conductance level change (2)̂mho/30 sec) and the event marks. Although these recorders were small and reliable they were limited to 24 hours of recording per tape. More importantly, the playback system was unacceptable. There was no computer interface. Data had to be manually scored from (very long) paper chart recordings.

The second system comprised the Oxford Medilog 8 channel cassette recorder and Oxford 9000 playback system. 149 hot flashes were recorded in 10 symptomatic postmenopausal women with a concordance of 77% between the skin conductance level criterion and event markers. However, these recorders were heavy (2 lbs), bulky (6×5×2 in.), and required an external skin conductance level circuit. Although the playback system had a computer interface, it was unstable for DC signals, such as skin conductance level, and required constant repair by Mr. Wasson.

The most recently used recorder, the Biolog 3991 x (UFI Biolog, Moro Bay, Calif.) provides some advantages over previous used systems: it is solid state, smaller (5×2×1 in), lighter (6 oz) and will record hot flashes for 7 days. With this recorder, a concordance of 70% between the skin conductance level criterion and event marks was obtained for waking hot flashes in 17 symptomatic breast cancer survivors. In a more recent study, an inventor herein trained 20 nurses to perform 4-day recordings in an inpatient setting on 60 symptomatic postmenopausal women using thirty Biolog recorders. Nurses were required to change the electrodes and gel every morning. Several problems were encountered with this system. Five recorders failed due to jamming of the event mark buttons. The initial setup was time consuming because the clock is difficult to program. Data scoring was cumbersome because two separate programs are needed and the programs are computationally inefficient.

However, the most limiting problem, which is common to all of the above systems, is the patient/device interface; that is, the electrodes and gel. It is not possible to wear any of these recorders in the shower or bathtub; therefore, the electrodes and recorder must be removed. After bathing, new electrodes with fresh gel must be properly applied and attached to the recorder, which must then be restarted. This process is much too complex for patients to perform, so they must return to the laboratory if extended recordings are needed. This is not sufficiently practical for clinical trials. An additional problem with skin conductance level measurements is that, in patients with many hot flashes the electrodes fill up with sweat. This creates severe artifacts and distorted, recordings.

### BRIEF DESCRIPTION OF THE DRAWING

Comprehension of the invention is facilitated by reading the following detailed description in conjunction with the annexed drawing, in which:

FIG. 1 is a plan representation of a miniature hygrometric sensor that is useful in the practice of the invention; and

FIG. 2 is a plan representation of a miniature recorder having a microcontroller, a flash memory, and a hearing aid battery.

### DETAILED DESCRIPTION

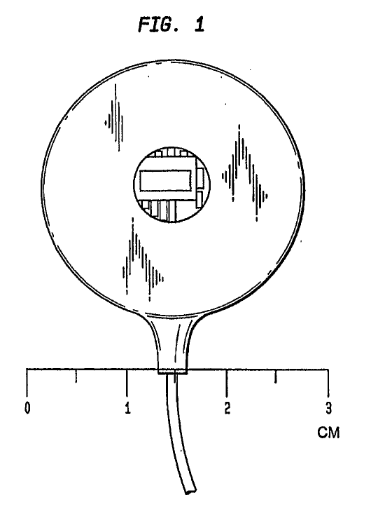

FIG. 1 is a plan representation of a miniature hygrometric sensor that is useful in the practice of the invention. The miniature hygrometric sensor is provided with a port, in the form of electrical leads (shown but not specifically designated), that provide electrical signals responsive to relative humidity.

In accordance with a first study that establishes the efficacy of the present system of treating hot flashes, the miniature hygrometric sensor was mounted within a 2.5 cm diameter plexiglass disc (not specifically designated). The sensor was attached over the sternum of a test subject (not shown) with standard, double-sided, adhesive collars (not shown). The output of the sensor was recorded using a conventional analog-to-digital converter (not shown) and a personal computer (not shown). Two 2.5 cm Ag/AgCl electrodes (not shown), of a type that are commercially available from Graphic Controls, Buffalo, N.Y., were filled with 0.05 M KCl gel and attached on either side of the miniature hygrometric sensor approximately 4 cm apart. The skin conductance level was recorded using a 0.5 volt constant-voltage circuit, A/D converter, and PC computer. A conventional event marker button was connected to the computer.

During this study, test subjects wore cotton scrub suits and reclined in a large arm chair, in a temperature and humidity-controlled (26° C., 50% relative humidity-RH) room. They were heated with two 40×60 cm circulating water pads at 42° C. Subjects were recorded for two hours between 1000 and 1700 h. They were instructed to press the event marker button each time a hot flash occurred. After the 2 h recording period the skin conductance level electrodes were removed. The sensor was connected to a Biolog skin conductance level recorder that had been modified to record signals from the sensor. Subjects were then sent home for twenty-four hours after being instructed not to permit the recorder to become wet and to press the event marker each time they had a hot flash.

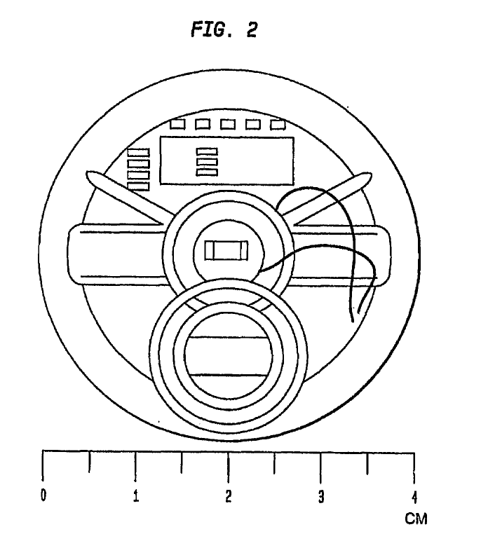

FIG. 2 is a plan representation of a recorder having a microcontroller, a flash memory, and a hearing aid battery. In this specific illustrative embodiment of the invention, the miniature recorder is 3.8 cm in diameter, 1 cm thick, and weighs 14 gm including the battery (not shown). The recorder was obtained from Kolar Engineering of Royal Oak, Mich. The test subjects in a second study were instrumented with the recorder, two skin conductance electrodes, and the event marker and received the heat test procedures described above with respect to the first study. At the end of the two-hour period, the skin conductance electrodes were attached to a Biolog skin conductance level recorder (not shown). Again, the test subjects were instructed not to permit the devices to become wet and to press the event marker each time a hot flash occurred. Nine women were then recorded for twenty-four hours, and one for sixty hours.

The data were then analyzed. With respect to the first study, the relative humidity, skin conductance level signals and event marks were downloaded from the laboratory personal computer. The amplitude and duration of each putative hot flash were scored using Excel (Microsoft, Redmond, Wash.). Then, in order to determine the criterion of relative humidity change for a hot flash, these data were analyzed with a receiver operating characteristic analysis using an skin conductance level change of 2 μmho/30 seconds as the gold standard. Then, the ambulatory relative humidity data were downloaded from the Biolog recorder and analyzed with a receiver operating characteristic analysis using the event marks as the gold standard.

With respect to the second study, the relative humidity data from the recorder was downloaded. The amplitude and duration of putative hot flashes were scored, and the skin conductance level signals from the Biolog recorder were downloaded and scored using Biolog software and the 2 μmho/30 seconds criterion.

A receiver operating characteristic analysis was employed to compare the Biolog and relative humidity detected hot flash counts using the Biolog as the gold standard. Percent relative humidity changes from 2%-6% in 0.5% steps over 1 minute and 2 minute intervals were examined. A second such analysis was performed using the event marks as the gold standard.

Twenty hot flashes meeting the 2 μmho/30 seconds skin conductance level criterion were recorded during the laboratory session of the first study. All were accompanied by an event mark and by a relative humidity increase of approximately 3% per minute. There were no false positive or false negative events. Thus, the positive predictive value, the sensitivity and the specificity for the 3%/minute relative humidity change vs. the skin conductance level criterion and the event marks are 100%.

The receiver operating characteristic analysis of the ambulatory data showed that a relative humidity increase of 3%/minute produced the best positive predictive value. There were an average of 12.6±2.6 standard deviation event marks per test subject and 17.6±2.2 relative humidity increases (3%/min) per test subject during these recordings. Using this criterion relative humidity change against the event marks as the gold standard, the positive predictive value was 71.6%, the specificity was 60.3%, and the sensitivity was 99%.

Eighteen hot flashes meeting the 2 μmho/30 seconds skin conductance level criterion were recorded during the laboratory session of the second study. All were accompanied by an event mark and by a relative humidity increase of 3%/minute. There were no false positive or false negative events. Thus the positive predictive value, the sensitivity and the specificity for the 3%/minute relative humidity change vs. the skin conductance level criterion and the event marks are 100%.

The receiver operating characteristic analysis of the ambulatory monitoring data again showed that a relative humidity increase of 3%/minute produced the best positive predictive value. There were an average of 23.8 (12.7 standard deviation) Biolog-detected hot flashes per test subject and 21.4 (11.9) flashes per test subject detected by relative humidity. Using the Biolog flashes as the gold standard, the positive predictive value for relative humidity was 95.6%, the specificity was 95.2% and the sensitivity was 90.9%.

There were an average 13.0 (9.9) event marks per test subject recorded on the Biolog recorder. Using these as the gold standard, the positive predictive value for relative humidity was 59.7% and the positive predictive value for the Biolog recorder (skin conductance level) was 52.8%.

There were no significant correlations (Pearson r and Spearman rho) between average daily humidity as reported by NOAA (range 60-100%) and any of the variables reported above. The ambient relative humidity on the chest was estimated by the basal values shown on the relative humidity recordings and ranged from 40-95%. There were no differences in hot flash detection rates across this range and the recordings did not plateau at the upper end.

It is highly advantageous that the miniature hygrometric hot flash recorder of the present invention uses neither electrodes nor gel. The recorder was, during the aforementioned first and second studies, well-tolerated by test subjects and did not fall off. Test subjects reported that they did not notice the presence of the recorder and found it vastly preferable to the Biolog recorder.

In the laboratory, the correspondence among a relative humidity increase of 3%/minute, skin conductance level-detected hot flashes, and test subject event marks was 100%. During twenty-four to sixty hour ambulatory monitoring, using skin conductance level-detected hot flashes as the gold standard, the positive predictive value for the relative humidity criterion was 95.6%, the specificity was 95% and the sensitivity was 90.9%. Thus, the relative humidity-recorded data were very similar to those recorded on the Biolog recorder, but used a simpler, smaller, and less obtrusive device. Using the event marks as the gold standard, the positive predictive value for relative humidity was superior to that of the Biolog recorder.

In the laboratory the positive predictive values of both devices compared to the event marks was a perfect, 100%. In the field the positive predictive values for both devices compared to the event marks was considerably worse although better than those previously reported for the Biolog recorder. This may be due to under-reporting of the hot flashes by the test subjects, as demonstrated in previous studies, and may be the result of distraction, inconvenience, or failure to perceive the hot flashes during sleep.

The relative humidity recordings were not significantly affected by wide ranges of outdoor humidity or by ambient humidity recorded on the chest. However, the device may not record accurately in locations where the relative humidity is near 100%, such as a steamy bathroom or some tropical climates.

Although the invention has been described in terms of specific embodiments and applications, persons skilled in the art may, in light of this teaching, generate additional embodiments without exceeding the scope or departing from the spirit of the invention described herein. Accordingly, it is to be understood that the drawing and description in this disclosure are proffered to facilitate comprehension of the invention, and should not be construed to limit the scope thereof.

What is claimed is:

1. A method of determining the presence of a hot flash of a patient, the method comprising the step of monitoring relative humidity.

2. The method of claim 1, wherein said step of monitoring relative humidity is performed in the vicinity of the sternum of a patient.

3. The method of claim 1, wherein said step of monitoring relative humidity comprises the further step of installing a transducer in the vicinity of the sternum of a patient, the transducer having an output port for producing electrical data responsive to relative humidity.

4. The method of claim 3, wherein said transducer comprises a miniature capacitance transducer.

5. The method of claim 3, wherein there is provided the further step of recording the electrical data produced by said transducer in a data recorder.

6. The method of claim 5, wherein there is further provided the step of analyzing the electrical data to determine a rate of change of the relative humidity.

7. The method of claim 6, wherein there is further provided the step of determining the presence of a hot flash in the patient in response to the rate of change of the relative humidity corresponding to an increase of approximately 3% per minute.

8. An apparatus for determining the occurrence of a hot flash of a patient, the apparatus comprising a transducer that measures relative humidity.

9. The apparatus of claim 8, wherein said transducer is a miniature capacitance transducer.

10. The apparatus of claim 8, wherein said capacitance transducer is of low weight and small size.

11. The apparatus of claim 10, wherein said capacitance transducer is dimensioned approximately 1.5 along a major axis.

12. The apparatus of claim 8, wherein there is further provided a computer arrangement for downloading hot flash event waveforms obtained from said transducer.

13. The apparatus of claim 12, wherein there is further provided a software system for determining a time rate of change of the hot flash event waveforms obtained from said transducer.
